(19) United States
(12) Patent Application Publication
(10) Pub. No.: US 2020/0085997 A1
(43) Pub. Date: Mar. 19, 2020

(54) SYNTHETIC TISSUE GLUE

(71) Applicants: Baxter International Inc., Deerfield, IL (US); Baxter Healthcare SA, Glattpark (Opfikon) (CH)

(72) Inventor: Haitao Qian, Buffalo Grove, IL (US)

(21) Appl. No.: 16/574,569

(22) Filed: Sep. 18, 2019

Related U.S. Application Data
(60) Provisional application No. 62/733,297, filed on Sep. 19, 2018.

(51) Int. Cl. A61L 24/04; A61B 17/00; C08L 87/00; A61L 24/00; C08L 71/02

(52) U.S. Cl. CPC A61L 24/043; A61B 17/00491; C08L 87/005; A61L 24/0042; C08L 2203/162; A61B 2017/00495; C08L 2201/06; C08L 2203/02; C08L 2205/025; C08L 71/02

(57) ABSTRACT

Compositions for use as a tissue glue or sealant are disclosed. The compositions include first and second precursor molecules, wherein the first precursor molecule is a polyoxyethylene-polyoxypropylene block copolymer having at least two nucleophilic groups and the second precursor molecule is a polyoxyethylene-polyoxypropylene block copolymer having at least two electrophilic groups. Biomaterials for use as a tissue glue or sealant, methods of making such biomaterials, devices for applying a biomaterial to a tissue of a patient, methods of sealing a tissue of a patient, and kits also are disclosed.

### CROSS-REFERENCE TO RELATED APPLICATIONS

This application claims the benefit of U.S. Provisional Patent Application No. 62/733,297, filed Sep. 19, 2018, the entire content of which is hereby incorporated by reference in its entirety.

### BACKGROUND

### Field

The disclosure is directed to biomaterials useful as tissue glues and sealants. More particularly, the present disclosure is directed to biomaterials, precursor molecules for forming biomaterials, systems for using biomaterials, and methods of making and using biomaterials.

### Description of the Related Art

In the medical field, tissue glues and sealants are used to seal or repair biological tissue. A number of tissue glues and sealants are formed by mixing two or more components that react to form a biomaterial having sufficient strength and adhesion for a desired application, such as to seal or repair dura or other tissue. The separate sealant components are preferably biocompatible, and can be absorbed by the body, or are otherwise harmless to the body, so that they do not require later removal. For example, fibrin-based tissue sealants are made from a combination of at least two primary components, fibrinogen and thrombin. Upon coming into contact with each other, the fibrinogen and thrombin components interact to form a crosslinked polymeric network of fibrin that is suitable as a tissue sealant. Crosslinked gelatin-based tissue glues are another example of a two-component sealant and typically are formed by crosslinking gelatin with an aldehyde such as formaldehyde or glutaraldehyde. Another example of a tissue glue are urethane-based tissue glues that use a lysine diisocyanate ethyl ester to form a crosslinked biomaterial.

Other crosslinked polymer compositions include two-component systems based on polyethylene glycol and are disclosed, for example, in U.S. Pat. No. 5,874,500, which is hereby incorporated by reference in its entirety.

### SUMMARY

In light of the disclosure herein, and without limiting the scope of the invention in any way, in a first aspect of the present disclosure, which may be combined with any other aspect listed herein unless specified otherwise, a composition includes first and second precursor molecules wherein: i) the first precursor molecule has a formula I or II:

A-[(C₂H₄O)ₘ₁—(C₃H₆O)ₙ₁-B]_{y} (I)

or

A-[(C₃H₆O)ₘ₂—(C₂H₄O)ₙ₂-B]_{y} (II)

wherein m1, m2, n1, and n2 are independently integers from 1 to 200; y is 2 or greater; A is a branch point; and B comprises a nucleophilic functional group; and ii) the second precursor molecule has a formula III or IV:

D-[(C₂H₄O)ₚ₁—(C₃H₆O)_{q1}-E]_{z} (III)

or

D-[(C₃H₆O)ₚ₂—(C₂H₄O)_{q2}-E]_{z} (IV)

wherein p1, p2, q1, and q2 are independently integers from 1 to 200; z is 2 or greater; D is a branch point; and E comprises an electrophilic functional group.

In a second aspect of the present disclosure, which may be combined with any other aspect listed herein unless specified otherwise, a dual compartment syringe includes a first precursor molecule loaded in a first compartment and a second precursor molecule loaded in a second compartment, wherein the first precursor molecule has a formula I or II as described herein, and the second precursor molecule has a formula III or IV as described herein.

In a third aspect of the present disclosure, which may be combined with any other aspect listed herein unless specified otherwise, the disclosure provides a biomaterial formed by reacting first and second precursor molecules wherein the first precursor molecule has a formula I or II as described herein, and the second precursor molecule has a formula III or IV as described herein.

In a fourth aspect of the present disclosure, which may be combined with any other aspect listed herein unless specified otherwise, a biomaterial includes a crosslinked polymeric network having a structure:

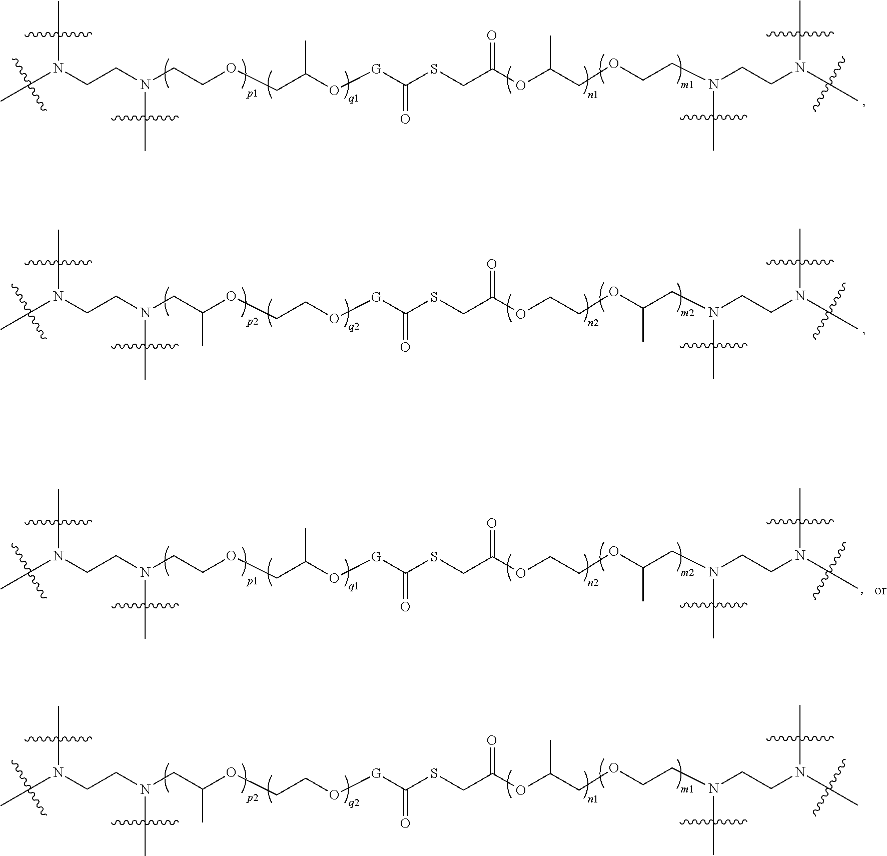

wherein m1, m2, n1, n2, p1, p2, q1, and q2 are independently integers from 1 to 200; G is absent or —CO(CH₂)ᵣ—; and r is an integer from 1 to 5.

In a fifth aspect of the present disclosure, which may be combined with any other aspect listed herein unless specified otherwise, a method of making a biomaterial includes reacting first and second precursor molecules to form a biomaterial in the form of a crosslinked three dimensional network, wherein the first precursor molecule has a formula I or II as described herein, and the second precursor molecule has a formula III or IV as described herein.

In a sixth aspect of the present disclosure, which may be combined with any other aspect listed herein unless specified otherwise, a method of sealing a tissue in a patient includes applying to a tissue in a patient a composition comprising first and second precursor molecules, wherein the first precursor molecule has a formula I or II as described herein, and the second precursor molecule has a formula III or IV as described herein.

In a seventh aspect of the present disclosure, which may be combined with any other aspect listed herein unless specified otherwise, a kit includes a first precursor molecule in a first container and a second precursor molecule in a second container, wherein the first precursor molecule has a formula I or II as described herein, and the second precursor molecule has a formula III or IV as described herein.

In light of the disclosure and aspects set forth herein, it is accordingly an advantage of the present disclosure to provide a biomaterial that is low-swelling and strong. Without wishing to be bound by theory, it is believed that by using a polyethylene oxide-polypropylene oxide block copolymer, the biomaterial is less swellable compared to a polyethylene oxide homopolymer having an otherwise similar number of monomer repeat units. Low-swelling tissue glues and sealants can be particularly advantageous in certain applications such as neural and spinal sealing.

It is another advantage of the present disclosure to control the curing or gelation time of the composition to the desired level by varying the pH of the composition. Without wishing to be bound by theory, it is believed that increasing the pH of the composition decreases the curing or gelation time.

It is a further advantage of the present disclosure to provide a synthetic biomaterial that does not contain proteins or animal products.

It is yet another advantage of the present disclosure to provide a biomaterial that is partially or fully bioresorbable.

Additional features and advantages of the disclosed formulations are described in, and will be apparent from, the following Detailed Description. The features and advantages described herein are not all-inclusive and, in particular, many additional features and advantages will be apparent to one of ordinary skill in the art in view of the description. Also, any particular embodiment does not necessarily have to have all of the advantages listed herein. Moreover, it should be noted that the language used in the specification has been principally selected for readability and instructional purposes, and not to limit the scope of the inventive subject matter.

### DETAILED DESCRIPTION

Certain embodiments described herein relate generally to the field of tissue glues and sealants. More particularly, some embodiments described herein relate to compositions comprising first and second precursor molecules. Related embodiments described herein relate to dual compartment syringes comprising such first and second precursor molecules loaded in the compartments of the dual compartment syringe. Additional related embodiments described herein relate to biomaterials formed by reaction of such first and second precursor molecules. Further related embodiments described herein relate to biomaterials comprising a crosslinked polymeric network. Related embodiments described herein also relate to methods of making biomaterials by reacting such first and second precursor molecules. Additional related embodiments described herein relate to methods of sealing a tissue in a patient by applying compositions comprising such first and second precursor molecules to the tissue in the patient. Further related embodiments described herein relate to kits comprising such first and second precursor molecules.

As used herein, the term “substantially free” may refer to a composition that contains no more than 1% of the specified component, for example, no more than 0.5%, no more than 0.1%, no more than 0.05%, no more than 0.02%, no more than 0.01%, no more than 0.005%, no more than 0.002%, and/or no more than 0.001% of the specified component. For example, a composition that is “substantially free of poly(ethylene oxide) derivatives” may refer to a composition that contains no more than 1% poly(ethylene oxide) derivatives, such as no more than 0.5%, no more than 0.1%, no more than 0.05%, no more than 0.02%, no more than 0.01%, no more than 0.005%, no more than 0.002%, and/or no more than 0.001% poly(ethylene oxide) derivatives.

As used herein, the term “poly(ethylene oxide) derivative” refers to a straight-chain or branched poly(ethylene oxide) homopolymer modified to contain one or more (e.g., two, three, four, six, or eight) nucleophilic or electrophilic groups.

### Compositions

The disclosure provides compositions for the manufacture of a biomaterial suitable for use as a tissue glue or sealant. The compositions contain at least first and second precursor molecules that are multifunctional (e.g., di-, tri-, or tetra-functional) for forming crosslinked networks, such as crosslinked polymeric networks. The precursor molecules can crosslink in situ at the site of need in a human or animal to form the polymeric network of the biomaterial.

The precursor molecules disclosed herein include a first precursor molecule containing at least two nucleophilic functional groups and a second precursor molecule containing at least two electrophilic functional groups. The nucleophilic groups of the first precursor molecule and the electrophilic groups of the second precursor molecule are selected such that the nucleophilic groups are capable of reacting with the electrophilic groups to form covalent linkages between the first and second precursor molecules, typically under physiological conditions or under basic conditions. This can be achieved by different reaction mechanisms, including a nucleophilic substitution reaction.

The first precursor molecules disclosed herein are polypropylene oxide-polyethylene oxide (PPO-PEO) block copolymers that contain at least two nucleophilic functional groups. In an embodiment of the present disclosure, which may be combined with any other embodiment listed herein unless specified otherwise, the first precursor molecule has a formula I or II:

A-[(C₂H₄O)ₘ₁—(C₃H₆O)ₙ₁-B]_{y} (I)

or

A-[(C₃H₆O)ₘ₂—(C₂H₄O)ₙ₂-B]_{y} (II)

In an embodiment of the present disclosure, which may be combined with any other embodiment listed herein unless specified otherwise, m1 and n1 of formula I are independently integers from 1 to 200, for example, m1 is an integer from 2 to 200, such as from 4 to 100, from 5 to 50, from 6 to 40, from 8 to 30, from 10 to 25, from 10 to 22, from 12 to 20, from 14 to 18, and/or 16; and n1 is an integer from 2 to 200, such as from 4 to 100, from 5 to 50, from 6 to 45, from 8 to 40, from 10 to 35, from 12 to 30, from 15 to 25, from 17 to 21, and/or 19.

In an embodiment of the present disclosure, which may be combined with any other embodiment listed herein unless specified otherwise, m2 and n2 of formula II are independently integers from 1 to 200, for example, m2 is an integer from 2 to 200, such as from 4 to 100, from 5 to 50, from 8 to 45, from 10 to 40, from 15 to 40, from 20 to 35, from 22 to 30, from 24 to 28, and/or 26; and n2 is an integer from 2 to 200, such as from 2 to 100, from 2 to 50, from 2 to 25, from 2 to 15, from 2 to 10, from 2 to 8, from 2 to 6, from 2 to 4, from 4 to 10, from 4 to 8, from 4 to 6, from 3 to 5, and/or 4.

In an embodiment of the present disclosure, which may be combined with any other embodiment listed herein unless specified otherwise, y of formulae I or II is 2 or greater, such as 2 to 8, 2 to 6, 2 to 4, 2, 3, or 4.

In an embodiment of the present disclosure, which may be combined with any other embodiment listed herein unless specified otherwise, A of formulae I or II is a branch point. In an embodiment of the present disclosure, which may be combined with any other embodiment listed herein unless specified otherwise, branch point A is selected from the group consisting of carbon, glycerol, pentaerythritol, dipentaerythritol, and ethylene diamine.

In an embodiment of the present disclosure, which may be combined with any other embodiment listed herein unless specified otherwise, B of formulae I or II comprises a nucleophilic functional group. In an embodiment of the present disclosure, which may be combined with any other embodiment listed herein unless specified otherwise, B comprises a thiol group or an amino group.

The first precursor molecules are multifunctional monomers, oligomers, and/or polymers. In an embodiment of the present disclosure, which may be combined with any other embodiment listed herein unless specified otherwise, the first precursor molecule has a number average molecular weight of about 2 kD to about 20 kD, such as about 3 kD to about 15 kD, about 4 kD to about 12 kD, about 5 kD to about 10 kD, about 6 kD to about 8 kD, about 7 kD to about 9 kD, and/or about 7.5 kD.

In an embodiment of the present disclosure, which may be combined with any other embodiment listed herein unless specified otherwise, the first precursor molecule is a 4-arm poly(ethylene oxide-polypropylene oxide) block copolymer of formula I or II, wherein A is an ethylene diamine molecule and B comprises a thiol group, such as a COCH₂SH group. Four-arm poly(ethylene oxide-polypropylene oxide) block copolymers with an ethylene diamine core molecule are sold, for example, by BASF under the tradename TETRONIC.

In an embodiment of the present disclosure, which may be combined with any other embodiment listed herein unless specified otherwise, the first precursor molecule has a formula:

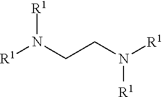

wherein each R¹ is

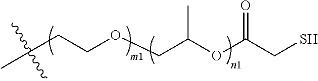

and m1 and n1 are independently integers from 1 to 200, for example, m1 is an integer from 2 to 200, such as from 4 to 100, from 5 to 50, from 6 to 40, from 8 to 30, from 10 to 25, from 10 to 22, from 12 to 20, from 14 to 18, and/or 16; and n1 is an integer from 2 to 200, such as from 4 to 100, from 5 to 50, from 6 to 45, from 8 to 40, from 10 to 35, from 12 to 30, from 15 to 25, from 17 to 21, and/or 19.

In an embodiment of the present disclosure, which may be combined with any other embodiment listed herein unless specified otherwise, the first precursor molecule has a formula:

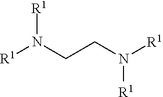

wherein each R¹ is

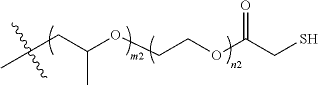

and m2 and n2 are independently integers from 1 to 200, for example, m2 is an integer from 2 to 200, such as from 4 to 100, from 5 to 50, from 8 to 45, from 10 to 40, from 15 to 40, from 20 to 35, from 22 to 30, from 24 to 28, and/or 26; and n2 is an integer from 2 to 200, such as from 2 to 100, from 2 to 50, from 2 to 25, from 2 to 15, from 2 to 10, from 2 to 8, from 2 to 6, from 2 to 4, from 4 to 10, from 4 to 8, from 4 to 6, from 3 to 5, and/or 4.

The second precursor molecules disclosed herein are polypropylene oxide-polyethylene oxide (PPO-PEO) block copolymers that contain at least two electrophilic functional groups. In an embodiment of the present disclosure, which may be combined with any other embodiment listed herein unless specified otherwise, the second precursor molecule has a formula III or IV:

D-[(C₂H₄O)ₚ₁—(C₃H₆O)_{q1}-E]_{z} (III)

or

D-[(C₃H₆O)ₚ₂—(C₂H₄O)_{q2}-E]_{z} (IV)

In an embodiment of the present disclosure, which may be combined with any other embodiment listed herein unless specified otherwise, p1 and q1 of formula III are independently integers from 1 to 200, for example, p1 is an integer from 2 to 200, such as from 4 to 100, from 5 to 50, from 6 to 40, from 8 to 30, from 10 to 25, from 10 to 22, from 12 to 20, from 14 to 18, and/or 16; and q1 is an integer from 2 to 200, such as from 4 to 100, from 5 to 50, from 6 to 45, from 8 to 40, from 10 to 35, from 12 to 30, from 15 to 25, from 17 to 21, and/or 19.

In an embodiment of the present disclosure, which may be combined with any other embodiment listed herein unless specified otherwise, p2 and q2 of formula IV are independently integers from 1 to 200, such as 10 to 200, 20 to 100, and/or 30 to 70, for example, p2 is an integer from 2 to 200, such as from 4 to 100, from 5 to 50, from 8 to 45, from 10 to 40, from 15 to 40, from 20 to 35, from 22 to 30, from 24 to 28, and/or 26; and q2 is an integer from 2 to 200, such as from 2 to 100, from 2 to 50, from 2 to 25, from 2 to 15, from 2 to 10, from 2 to 8, from 2 to 6, from 2 to 4, from 4 to 10, from 4 to 8, from 4 to 6, from 3 to 5, and/or 4.

In an embodiment of the present disclosure, which may be combined with any other embodiment listed herein unless specified otherwise, z of formulae III or IV is 2 or greater, such as 2 to 8, 2 to 6, 2 to 4, 2, 3, or 4.

In an embodiment of the present disclosure, which may be combined with any other embodiment listed herein unless specified otherwise, D of formulae III or IV is a branch point. In an embodiment of the present disclosure, which may be combined with any other embodiment listed herein unless specified otherwise, branch point D is selected from the group consisting of carbon, glycerol, pentaerythritol, dipentaerythritol, and ethylene diamine.

In an embodiment of the present disclosure, which may be combined with any other embodiment listed herein unless specified otherwise, E of formulae III or IV comprises an electrophilic functional group. Suitable electrophilic groups include, but are not limited to, mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters (e.g., succinimidyl carbonate, succinimidyl glutarate, succinimidyl acetate, succinimidyl succinimide), N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; isocyanates; maleimides; substituted maleimides; haloalkanes; epoxides; imines; aziridines; olefins (including conjugated olefins) such as vinyl sulfone, ethenesulfonyl, etheneimino, acrylate, methacrylate, and α,β-unsaturated aldehydes and ketones; and combinations thereof. In an embodiment of the present disclosure, which may be combined with any other embodiment listed herein unless specified otherwise, E comprises a succinimidyl ester group. In an embodiment of the present disclosure, which may be combined with any other embodiment listed herein unless specified otherwise, E is:

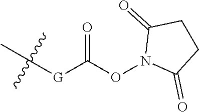

wherein G is absent or —CO(CH₂)ᵣ—, and r is an integer from 1 to 5. In an embodiment of the present disclosure, which may be combined with any other embodiment listed herein unless specified otherwise, G is absent. In an embodiment of the present disclosure, which may be combined with any other embodiment listed herein unless specified otherwise, G is —CO(CH₂)ᵣ— and r is an integer from 2 to 4, such as 3.

The second precursor molecules are multifunctional monomers, oligomers, and/or polymers. In an embodiment of the present disclosure, which may be combined with any other embodiment listed herein unless specified otherwise, the second precursor molecule has a number average molecular weight of about 2 kD to about 20 kD, such as about 3 kD to about 15 kD, about 4 kD to about 12 kD, about 5 kD to about 10 kD, about 6 kD to about 8 kD, about 7 kD to about 9 kD, and/or about 8 kD.

In an embodiment of the present disclosure, which may be combined with any other embodiment listed herein unless specified otherwise, the second precursor molecule is a 4-arm poly(ethylene oxide-polypropylene oxide) block copolymer of formula III or IV, wherein D is an ethylene diamine molecule and E comprises a succinimidyl group, such as a

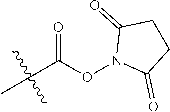 [SMILES: *C(=O)ON1C(=O)CCC1=O]

group or

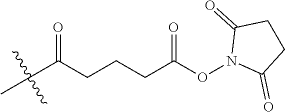 [SMILES: *C(=O)CCCC(=O)ON1C(=O)CCC1=O]

group. Four-arm poly(ethylene oxide-polypropylene oxide) block copolymers with an ethylene diamine core molecule are sold, for example, by BASF under the tradename TETRONIC.

In an embodiment of the present disclosure, which may be combined with any other embodiment listed herein unless specified otherwise, the second precursor molecule has a formula:

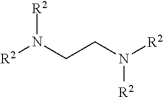

wherein each R² is

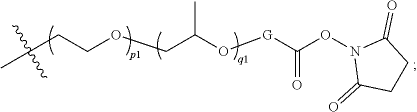

p1 and q1 are independently integers from 1 to 200, for example, p1 is an integer from 2 to 200, such as from 4 to 100, from 5 to 50, from 6 to 40, from 8 to 30, from 10 to 25, from 10 to 22, from 12 to 20, from 14 to 18, and/or 16; and q1 is an integer from 2 to 200, such as from 4 to 100, from 5 to 50, from 6 to 45, from 8 to 40, from 10 to 35, from 12 to 30, from 15 to 25, from 17 to 21, and/or 19; G is absent or —CO(CH₂)ᵣ—; and r is an integer from 1 to 5. In an embodiment of the present disclosure, which may be combined with any other embodiment listed herein unless specified otherwise, G is absent. In an embodiment of the present disclosure, which may be combined with any other embodiment listed herein unless specified otherwise, G is —CO(CH₂)ᵣ— and r is an integer from 2 to 4, such as 3.

In an embodiment of the present disclosure, which may be combined with any other embodiment listed herein unless specified otherwise, the second precursor molecule has a formula:

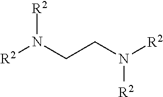

wherein each R² is

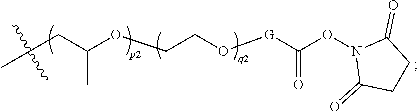

p2 and q2 are independently integers from 1 to 200, for example, p2 is an integer from 2 to 200, such as from 4 to 100, from 5 to 50, from 8 to 45, from 10 to 40, from 15 to 40, from 20 to 35, from 22 to 30, from 24 to 28, and/or 26; and q2 is an integer from 2 to 200, such as from 2 to 100, from 2 to 50, from 2 to 25, from 2 to 15, from 2 to 10, from 2 to 8, from 2 to 6, from 2 to 4, from 4 to 10, from 4 to 8, from 4 to 6, from 3 to 5, and/or 4; G is absent or —CO(CH₂)ᵣ—; and r is an integer from 1 to 5. In an embodiment of the present disclosure, which may be combined with any other embodiment listed herein unless specified otherwise, G is absent. In an embodiment of the present disclosure, which may be combined with any other embodiment listed herein unless specified otherwise, G is —CO(CH₂)ᵣ— and r is an integer from 2 to 4, such as 3.

The first and second precursor molecules are typically provided in the compositions described herein in a ratio such that a majority of the functional groups of both components react with their respective counterparts. In an embodiment of the present disclosure, which may be combined with any other embodiment listed herein unless specified otherwise, the ratio of functional groups of the first precursor molecule to functional groups of the second precursor molecule (i.e., the ratio of nucleophilic functional groups to electrophilic functional groups) is in the range of about 0.7 to about 1.3, such as about 0.8 to about 1.2, about 0.9 to about 1.1, and/or about 1 to 1.

The nucleophilic groups of the first precursor molecule and the electrophilic groups of the second precursor molecule typically react with each other under physiological conditions or under basic conditions. In an embodiment of the present disclosure, which may be combined with any other embodiment listed herein unless specified otherwise, the composition comprising the first and second precursor molecules further comprises a base, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the composition comprising first and second precursor molecules as described herein has a pH of about 4 to about 12, such as about 4 to about 11, about 4 to about 10, about 4 to about 9, about 4 to about 8, about 5 to about 7, about 5 to about 6, about 7 to about 12, about 8 to about 11, about 9 to about 10, about 7 to about 8, about 8 to about 9, about 9 to about 10, and/or about 10 to about 11. In an embodiment of the present disclosure, which may be combined with any other embodiment listed herein unless specified otherwise, the composition comprising first and second precursor molecules as described herein has a pH of about 4 to about 8, about 5 to about 7, and/or about 5 to about 6, and the first precursor molecule comprises an amino group. In an embodiment of the present disclosure, which may be combined with any other embodiment listed herein unless specified otherwise, the composition comprising first and second precursor molecules as described herein has a pH of about 8 to about 11, about 9 to about 10, and/or about 8 to about 9, and the first precursor molecule comprises a thiol group.

In an embodiment of the present disclosure, which may be combined with any other embodiment listed herein unless specified otherwise, the composition is free or substantially free of poly(ethylene oxide) derivatives.

### Devices

The disclosure provides devices to apply a biomaterial to a human or animal for use as a tissue glue or sealant. The devices comprise first and second precursor molecules as described herein, particularly first precursor molecules of formulae I and/or II, and second precursor molecules of formulae III and/or IV as described in the foregoing “Compositions” section. In an embodiment of the present disclosure, which may be combined with any other embodiment listed herein unless specified otherwise, a dual compartment syringe comprises the first precursor molecule loaded in a first compartment of the syringe and the second precursor molecule loaded in a second compartment of the syringe. A suitable dual compartment syringe typically includes first and second compartments (or barrels), a plunger, and optionally a syringe cap. An example of a dual compartment syringe device is described in U.S. Pat. No. 4,359,049, which is hereby incorporated by reference in its entirety.

In an embodiment of the present disclosure, which may be combined with any other embodiment listed herein unless specified otherwise, the first and second precursor molecules are present in the dual compartment syringe in liquid form, either neat (i.e., in the absence of any added solvent) or in solution (e.g., in a non-volatile organic solvent or a volatile organic solvent, which typically is inert, physiologically acceptable, and/or non-toxic, examples of which include, but are not limited to, dimethyl sulfoxide (DMSO) and fluorocarbon solvents).

The nucleophilic groups of the first precursor molecule and the electrophilic groups of the second precursor molecule typically react with each other under physiological conditions or under basic conditions. In an embodiment of the present disclosure, which may be combined with any other embodiment listed herein unless specified otherwise, the first compartment, the second compartment, or both the first and second compartments further include a base, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the first compartment, the second compartment, or both the first and second compartments further include a weak base, such as carbonate, N-methyldiethanolamine, 3-dimethylamino-1-propanol, and/or 2-dimethylamino-ethanol. In an embodiment of the present disclosure, which may be combined with any other embodiment listed herein unless specified otherwise, the first compartment further includes a weak base, such as carbonate, N-methyldiethanolamine, 3-dimethylamino-1-propanol, and/or 2-dimethylamino-ethanol. In an embodiment of the present disclosure, which may be combined with any other embodiment listed herein unless specified otherwise, each compartment including a weak base has a pH of about 7 to about 12, such as about 8 to about 11, about 9 to about 10, about 7 to about 8, about 8 to about 9, about 9 to about 10, and/or about 10 to about 11.

In an embodiment of the present disclosure, which may be combined with any other embodiment listed herein unless specified otherwise, the first and second compartments are free or substantially free of poly(ethylene oxide) derivatives.

In an embodiment of the present disclosure, which may be combined with any other embodiment listed herein unless specified otherwise, the dual compartment syringe is connected to a mixing device such as a static mixer for mixing the first precursor molecule with the second precursor molecule. The first precursor molecule can then be extruded into the mixing device, while the second precursor molecule is simultaneously extruded into the mixing device. Within the mixing device, the first and the second precursor molecules are mixed together to thereby form a reactive mixture. Thereafter, the reactive mixture can be extruded through an orifice and onto a surface (e.g., tissue), where a biomaterial is formed, which can function as a tissue glue or sealant, or the like. The reactive mixture begins forming a three-dimensional matrix immediately upon being formed by the mixing of the first and second precursor molecules in the mixing device. Accordingly, the reactive mixture is preferably extruded from the mixing device onto the tissue very quickly after it is formed so that the three-dimensional matrix forms on, and is able to adhere to, the tissue.

### Biomaterials

The disclosure provides biomaterials for use as a tissue glue or sealant in a human or animal. In an embodiment of the present disclosure, which may be combined with any other embodiment listed herein unless specified otherwise, the disclosure provides a biomaterial formed by reacting first and second precursor molecules as described herein, particularly first precursor molecules of formulae I and/or II, and second precursor molecules of formulae III and/or IV as described in the foregoing “Compositions” section.

In an embodiment of the present disclosure, which may be combined with any other embodiment listed herein unless specified otherwise, a biomaterial is provided comprising a crosslinked polymeric network having a structure:

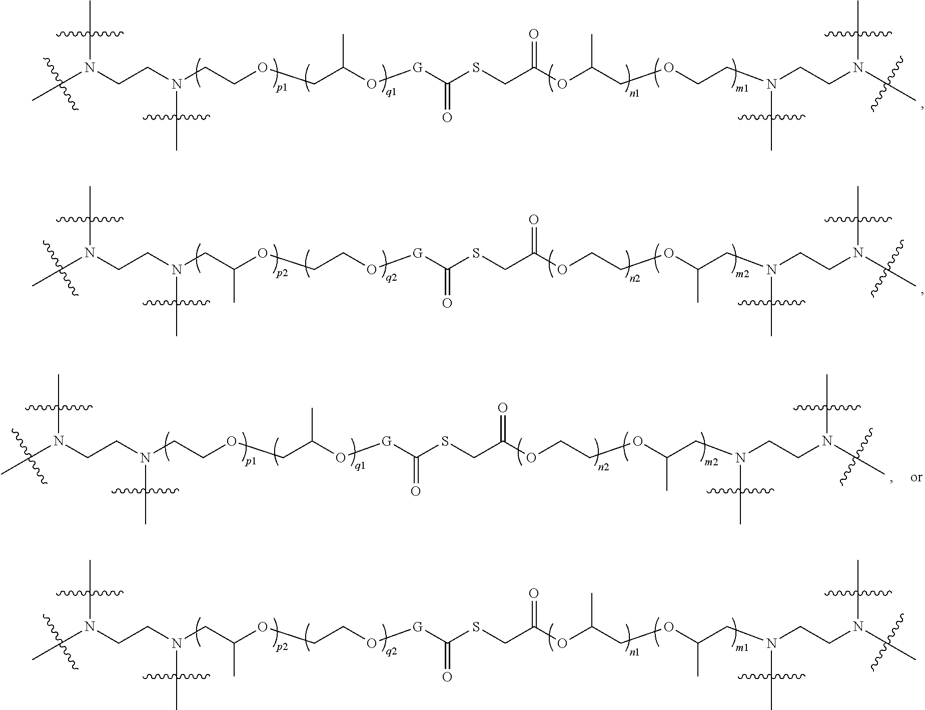

wherein m1, m2, n1, n2, p1, p2, q1, and q2 are independently integers from 1 to 200, including any of the ranges and values described in the foregoing “Compositions” section; G is absent or —CO(CH₂)ᵣ—; and r is an integer from 1 to 5. In the formulae above, use of the symbol [symbol] indicates that the molecular structure beyond this point is unspecified crosslinked material generally formed by reaction of the first and second precursor molecules as described herein. In an embodiment of the present disclosure, which may be combined with any other embodiment listed herein unless specified otherwise, G is absent. In an embodiment of the present disclosure, which may be combined with any other embodiment listed herein unless specified otherwise, G is —CO(CH₂)ᵣ— and r is an integer from 2 to 4, such as 3.

In an embodiment of the present disclosure, which may be combined with any other embodiment listed herein unless specified otherwise, the disclosure provides a method of making a biomaterial comprising reacting first and second precursor molecules as described herein, particularly first precursor molecules of formulae I and/or II, and second precursor molecules of formulae III and/or IV as described in the foregoing “Compositions” section to form a biomaterial in the form of a crosslinked three dimensional network. In an embodiment of the present disclosure, which may be combined with any other embodiment listed herein unless specified otherwise, the method comprises reacting the first and second precursor molecules in the presence of a base, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the method comprises reacting first and second precursor molecules under basic conditions, such as at a pH of about 7 to about 12, about 8 to about 11, about 9 to about 10, about 7 to about 8, about 8 to about 9, about 9 to about 10, and/or about 10 to about 11.

### Methods of Use

The compositions disclosed herein can be used in a variety of different applications. In general, these compositions can be adapted for use in any tissue engineering application where synthetic gel matrices are currently being utilized. For example, the compositions are useful as tissue glues and sealants, vascular sealants, in tissue augmentation, in tissue repair, as hemostatic agents, and in preventing tissue adhesions, and may be used in a variety of open, endoscopic, and laparoscopic surgical procedures. One of skill in the art can easily determine the appropriate administration protocol to use with any particular composition having a known gel strength and gelation time.

In one application, the compositions described herein can be used for medical conditions that require a coating or sealing layer to prevent the leakage of gases, liquid or solids.

Methods of use typically entail applying the composition to the damaged tissue or organ to seal 1) vascular and or other tissues or organs to stop or minimize the flow of blood; 2) thoracic tissue to stop or minimize the leakage of air; 3) gastrointestinal tract or pancreatic tissue to stop or minimize the leakage of fecal or tissue contents; 4) bladder or urethra to stop or minimize the leakage of urine; 5) dura to stop or minimize the leakage of cerebrospinal fluid; and/or 6) skin or serosal tissue to stop the leakage of serosal fluid. These compositions may also be used to adhere tissues together such as small vessels, nerves or dermal tissue. The compositions can be used 1) by applying them to the surface of one tissue and then a second tissue may be rapidly pressed against the first tissue or 2) by bringing the tissues in close juxtaposition and then applying the compositions such that both the first and second tissues are contacted with the compositions. In addition, the compositions can be used to fill spaces in soft and hard tissues that are created by disease or surgery. The compositions may additionally be used for anchoring or affixing surgical implants, such as in prosthetic fixation to, e.g., fix a mesh to tissue during hernia repair.

In an embodiment of the present disclosure, which may be combined with any other embodiment listed herein unless specified otherwise, the disclosure provides a method of sealing a tissue in a patient comprising applying to a tissue in a patient a composition comprising first and second precursor molecules as described herein, particularly first precursor molecules of formulae I and/or II, and second precursor molecules of formulae III and/or IV as described in the foregoing “Compositions” section.

### Kits

The disclosure provides kits for making a biomaterial for use as a tissue glue or sealant. Precursor molecules can be packaged in kits, typically along with written or otherwise illustrated instructions for use. The kits comprise first and second precursor molecules as described herein, particularly first precursor molecules of formulae I and/or II, and second precursor molecules of formulae III and/or IV as described in the foregoing “Compositions” section. In an embodiment of the present disclosure, which may be combined with any other embodiment listed herein unless specified otherwise, a kit comprises the first precursor molecule in a first container and the second precursor molecule in a second container. Typically, each component of the kit is packaged separately such that the component remains sterile and does not contact another component. For example, kits comprising a dual compartment syringe and at least one applicator may include a first sterile packaging containing the dual compartment syringe able to be delivered to the sterile field as well as a second sterile packaging containing the applicator, optionally with a static mixer.

In an embodiment of the present disclosure, which may be combined with any other embodiment listed herein unless specified otherwise, the first and second precursor molecules are present in the kit in liquid form. In an embodiment of the present disclosure, which may be combined with any other embodiment listed herein unless specified otherwise, the first and second precursor molecules are present in the kit in powder form.

The nucleophilic groups of the first precursor molecule and the electrophilic groups of the second precursor molecule typically react with each other under physiological conditions or under basic conditions. In an embodiment of the present disclosure, which may be combined with any other embodiment listed herein unless specified otherwise, the first container, the second container, or both the first and second containers further include a base, such as weak inorganic bases and weak organic bases, including, but not limited to carbonate, tertiary amines, tertiary alkylamines, N-methyldiethanolamine, 3-dimethylamino-1-propanol, 2-dimethylamino-ethanol, tributylamine, triethylamine, ethyldiisopropylamine, and N,N-dimethylbutylamine. In an embodiment of the present disclosure, which may be combined with any other embodiment listed herein unless specified otherwise, the first container, the second container, or both the first and second containers further include a weak base, such as carbonate, N-methyldiethanolamine, 3-dimethylamino-1-propanol, and/or 2-dimethylamino-ethanol. In an embodiment of the present disclosure, which may be combined with any other embodiment listed herein unless specified otherwise, the first container further includes a weak base, such as carbonate, N-methyldiethanolamine, 3-dimethylamino-1-propanol, and/or 2-dimethylamino-ethanol. In an embodiment of the present disclosure, which may be combined with any other embodiment listed herein unless specified otherwise, each container including a weak base has a pH of about 7 to about 12, such as about 8 to about 11, about 9 to about 10, about 7 to about 8, about 8 to about 9, about 9 to about 10, and/or about 10 to about 11. In an embodiment of the present disclosure, which may be combined with any other embodiment listed herein unless specified otherwise, each container including first or second precursor molecules in powder form and also including a weak base, when dissolved in a solvent, has a pH of about 7 to about 12, such as about 8 to about 11, about 9 to about 10, about 7 to about 8, about 8 to about 9, about 9 to about 10, and/or about 10 to about 11.

In an embodiment of the present disclosure, which may be combined with any other embodiment listed herein unless specified otherwise, the first and second containers are free or substantially free of poly(ethylene oxide) derivatives.

In an embodiment of the present disclosure, which may be combined with any other embodiment listed herein unless specified otherwise, the kit further comprises a dual compartment syringe as described herein wherein the first container is a first compartment of the dual compartment syringe and the second container is a second compartment of the dual compartment syringe. In an embodiment of the present disclosure, which may be combined with any other embodiment listed herein unless specified otherwise, the dual compartment syringe is present inside sterile packaging such as a sterile pouch or a sterile packet. In an embodiment of the present disclosure, which may be combined with any other embodiment listed herein unless specified otherwise, the kit further comprises one or more applicators present inside sterile packaging such as a sterile pouch or a sterile packet. In an embodiment of the present disclosure, which may be combined with any other embodiment listed herein unless specified otherwise, the kit further comprises a static mixer. In an embodiment of the present disclosure, which may be combined with any other embodiment listed herein unless specified otherwise, the kit further comprises a third container comprising a solvent.

Suitable kits are not limited to the devices described herein and may also include any other suitable delivery device known in the art.

### EXAMPLES

### Example 1

### Synthesis of NHS-Carbonate of Ethylenediamine Tetrakis(ethoxylate-block-propoxylate) Tetrol

TETRONIC 90R4 NHS carbonate was prepared as shown in Scheme 1 using TETRONIC 90R4 ethylenediamine tetrakis(ethoxylate-block-propoxylate) tetrol, average Mₙ ˜7,200, as the starting material. In particular, 21.60 g of TETRONIC 90R4 was azeotropically distilled with 250 ml of toluene overnight and the toluene was then removed under vacuum. Next, 25 ml of dry acetonitrile and 920 mg (6.6 mmol) of anhydrous potassium carbonate were added. The mixture was stirred while cooled in icy water for 1 hour after which 4.61 g of di(N-succinimidyl) carbonate was added. The mixture was stirred at 0° C. initially and the temperature was later raised to 45° C., and an additional 7.61 g of di(N-succinimidyl) carbonate was added during the reaction. After 6 days of reaction, the acetonitrile was removed under vacuum, after which 200 ml of MTBE was added to the reaction mixture. The mixture was shaken, let to settle for 1 hour, and then filtered. MTBE was removed under vacuum and 150 ml of toluene was added to the raw product. Hexane was added to saturate the solid. The mixture was filtered and he filtrate was concentrated under vacuum. The product was dissolved in 550 ml of dichloromethane and washed with 5% sodium bicarbonate (100 ml each, 3 times) and brine (100 ml each, 3 times) and then dried over sodium sulfate and potassium carbonate. After filtration, the solvent was removed under vacuum to yield the TETRONIC 90R4 NHS carbonate product.

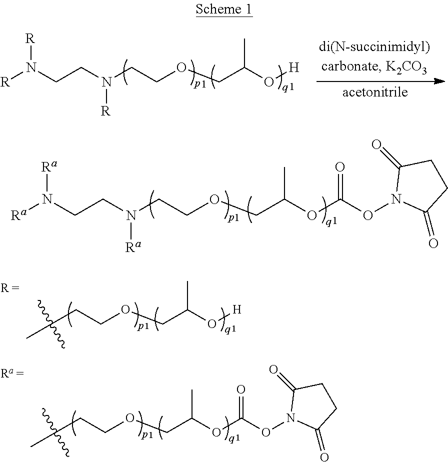

### Example 2

### Synthesis of Thioglycolate of Ethylenediamine Tetrakis(ethoxylate-block-propoxylate) Tetrol

TETRONIC 90R4 thiolglycolate was prepared as shown in Scheme 2 using TETRONIC 90R4 ethylenediamine tetrakis(ethoxylate-block-propoxylate) tetrol, average Mₙ ˜7,200, as the starting material. In particular, 421.60 g of TETRONIC 90R4 and 250 ml of toluene were refluxed for 22 hours in a 500 ml flask together with 1.50 g of p-toluenesulfonic acid and 22.00 g of thioglycolic acid under argon protection using a Dean-Stark distillation apparatus to collect the azeotropic water. Toluene was then removed under vacuum at 40-85° C. The obtained oil was dissolved in 500 ml of dichloromethane and washed with saturated sodium bicarbonate. The extract was further washed with brine (during washing, pH was adjusted with concentrated hydrochloric acid to 5.5) and dried over sodium sulfate. The solvent was removed under vacuum to yield the TETRONIC 90R4 thiolglycolate product.

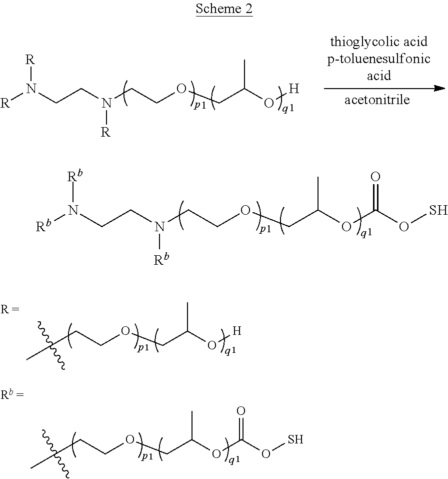

### Example 3

### Preparation of Synthetic Tissue Glue

A crosslinked synthetic tissue glue/sealant was prepared as shown in Scheme 3 by reacting the TETRONIC 90R4 NHS carbonate prepared in Example 1 with the TETRONIC 90R4 thiolglycolate prepared in Example 2. In particular, TETRONIC 90R4 NHS carbonate and TETRONIC 90R4 thiolglycolate were mixed using a dual syringe fixed with a static mixer. The two polymers reacted slowly to form the tissue glue, which was resilant, elastic, and pliable.

The reaction was accelerated when carried out at basic pH due to the presence of N-methyldiethanolamine or 3-dimethylamino-1-propanol.

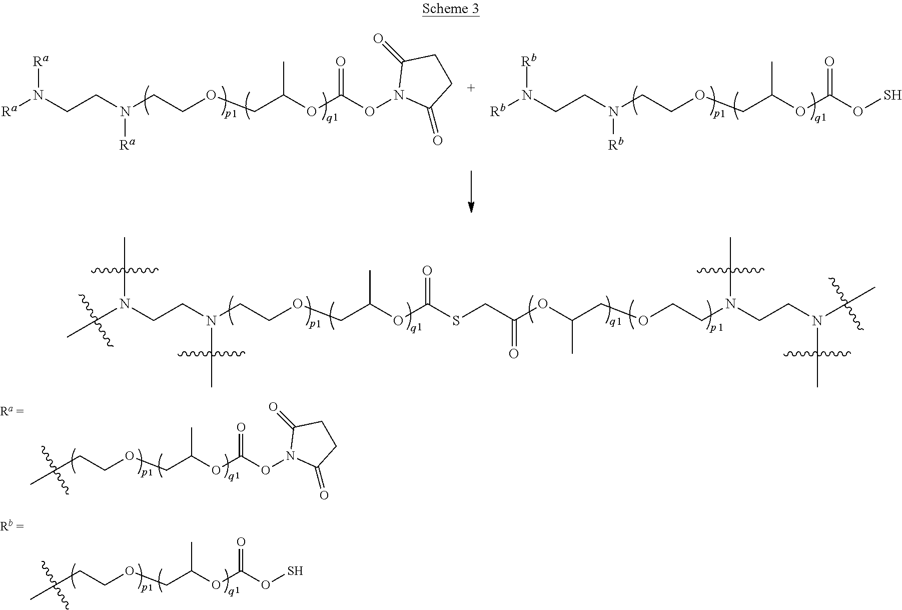

While the above is a complete description of the preferred embodiments of the invention, various alternatives, modifications, and equivalents may be used. Therefore, the above description should not be taken as limiting the scope of the invention which is defined by the appended claims.

1: A composition comprising first and second precursor molecules, wherein:
i) the first precursor molecule has a formula I or II:
A-[(C₂H₄O)ₘ₁—(C₃H₆O)ₙ₁-B]_{y} (I)
or
A-[(C₃H₆O)ₘ₂—(C₂H₄O)ₙ₂-B]_{y} (II)
wherein m1, m2, n1, and n2 are independently integers from 1 to 200;
y is 2 or greater;
A is a branch point; and
B comprises a nucleophilic functional group;
ii) the second precursor molecule has a formula III or IV:
D-[(C₂H₄O)ₚ₁—(C₃H₆O)_{q1}-E]_{z} (III)
or
D-[(C₃H₆O)ₚ₂—(C₂H₄O)_{q2}-E]_{z} (IV)
wherein p1, p2, q1, and q2 are independently integers from 1 to 200;
z is 2 or greater;
D is a branch point; and
E comprises an electrophilic functional group.

2: The composition of claim 1, wherein y is 2 to 8 and z is 2 to 8.

3-5. (canceled)

6: The composition of claim 1, wherein the branch point A is selected from the group consisting of carbon, glycerol, pentaerythritol, dipentaerythritol, and ethylene diamine, and the branch point D is selected from the group consisting of carbon, glycerol, pentaerythritol, dipentaerythritol, and ethylene diamine.

7. (canceled)

8: The composition of claim 1, wherein B comprises a thiol group or an amino group.

9: The composition of claim 1, wherein E comprises a succinimidyl ester group.

10: The composition of claim 1, wherein E is: 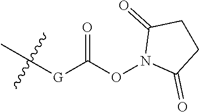
wherein G is absent or —CO(CH₂)ᵣ—; and
r is an integer from 1 to 5.

11-14. (canceled)

15: The composition of claim 1, wherein the first precursor molecule has a formula: 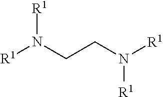
wherein each R¹ is 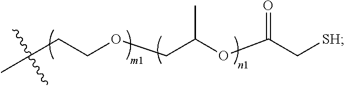
and
m1 and n1 are as defined in claim 1.

16: The composition of claim 1, wherein the first precursor molecule has a formula: 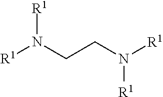
wherein each R¹ is 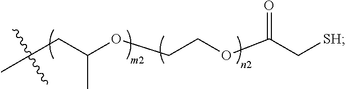
and
m2 and n2 are as defined in claim 1.

17: The composition of claim 1, wherein the second precursor molecule has a formula: 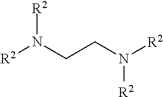
wherein each R² is 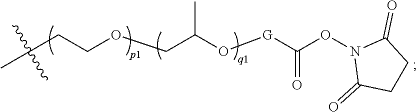
p1 and q1 are as defined in claim 1;
G is absent or —CO(CH₂)ᵣ—; and
r is an integer from 1 to 5.

18: The composition of claim 1, wherein the second precursor molecule has a formula: 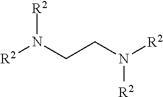
wherein each R² is 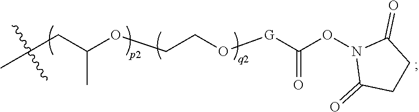
p2 and q2 are as defined in claim 1;
G is absent or —CO(CH₂)ᵣ—; and
r is an integer from 1 to 5.

19-21. (canceled)

22: The composition of claim 1, wherein the composition is substantially free of poly(ethylene oxide) derivatives.

23: A dual compartment syringe comprising the first and second precursor molecules of claim 1, where the first precursor molecule is loaded in a first compartment and the second precursor molecule is loaded in a second compartment.

24-47. (canceled)

48: The dual compartment syringe of claim 23, further comprising a mixing device.

49: A biomaterial formed by reacting the first and second precursor molecules of claim 1.

50: A biomaterial comprising a crosslinked polymeric network having a structure: 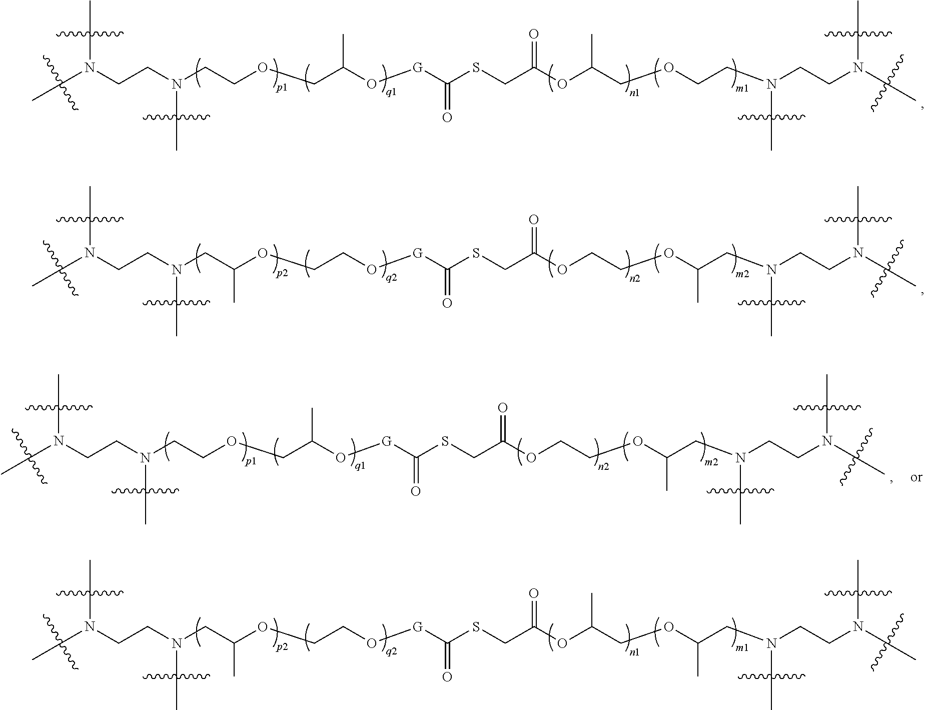
wherein m1, m2, n1, n2, p1, p2, q1, and q2 are independently integers from 1 to 200;
G is absent or —CO(CH₂)ᵣ—; and
r is an integer from 1 to 5.

51: A method of making a biomaterial comprising:
reacting first and second precursor molecules to form a biomaterial, wherein
i) the first precursor molecule has a formula I or II:
A-[(C₂H₄O)ₘ₁—(C₃H₆O)ₙ₁-B]_{y} (I)
or
A-[(C₃H₆O)ₘ₂—(C₂H₄O)ₙ₂-B]_{y} (II)
wherein m1, m2, n1, and n2 are independently integers from 1 to 200;
y is 2 or greater;
A is a branch point; and
B comprises a nucleophilic functional group;
ii) the second precursor molecule has a formula III or IV:
D-[(C₂H₄O)ₚ₁—(C₃H₆O)_{q1}-E]_{z} (III)
or
D-[(C₃H₆O)ₚ₂—(C₂H₄O)_{q2}-E]_{z} (IV)
wherein p1, p2, q1, and q2 are independently integers from 1 to 200;
z is 2 or greater;
D is a branch point; and
E comprises an electrophilic functional group; and
the biomaterial is in the form of a crosslinked three dimensional network.

52: The method of claim 51, wherein the first and second precursor molecules are reacted under basic conditions.

53: The method of claim 51, wherein the first and second precursor molecules are reacted in the presence of a weak base.

54: A method of sealing a tissue in a patient, comprising:
applying to a tissue in a patient the composition of claim 1.

55: A kit comprising the first and second precursor molecules of claim 1, wherein the first precursor molecule is in a first container and the second precursor molecule is in a second container.

56-66. (canceled)
